(19) United States
(12) Patent Application Publication
(10) Pub. No.: US 2024/0158337 A1
(43) Pub. Date: May 16, 2024

(54) GAS-PHASE OXIDATIVE COUPLING OF ALCOHOLS AND AMINES USING HETEROGENEOUS CATALYSTS

(71) Applicant: The Board of Trustees of The University of Alabama, Tuscaloosa, AL (US)

(72) Inventors: James William Shogren-Harris, Tuscaloosa, AL (US); Alexander Paul Minne, Tuscaloosa, AL (US)

(21) Appl. No.: 18/505,797

(22) Filed: Nov. 9, 2023

Related U.S. Application Data
(60) Provisional application No. 63/383,369, filed on Nov. 11, 2022.

(51) Int. Cl. C07C 231/10; B01J 23/44; B01J 23/52

(52) U.S. Cl. CPC C07C 231/10; B01J 23/44; B01J 23/52; B01J 2523/19; B01J 2523/824; C07C 2523/44; C07C 2523/52

(57) ABSTRACT

Disclosed are methods of forming an N,N-substituted-formamide comprising contacting a feed gas comprising an alcohol and an N,N-substituted-amine with a solid heterogeneous catalyst in the presence of an oxidizing agent at a reaction temperature, wherein the solid heterogeneous catalyst comprises one or more noble metals; and oxidatively coupling the feed gas under conditions effective to form an effluent gas comprising the N,N-substituted-formamide. Also disclosed herein are methods of forming a carbamide and/or a carbamate comprising contacting a feed gas comprising an alcohol and an N,N-substituted-amine with a solid heterogeneous catalyst in the presence of an oxidizing agent at a temperature greater than or equal to 155° C., wherein the solid heterogeneous catalyst comprises one or more noble metals; and oxidatively coupling the feed gas under conditions effective to form a product gas comprising the carbamide and/or carbamate.

### RELATED APPLICATION

This U.S. non-provisional application claims priority to, and the benefit of, U.S. Provisional Patent Application No. 63/383,369, filed Nov. 11, 2022, which is incorporated by reference herein in its entirety.

### BACKGROUND

Amide synthesis is estimated to be the most common reaction occurring in biological systems as well as pharmaceutical manufacturing, accounting for approximately 16% of all reactions in the manufacturing of modern medicine, and 25% of the drugs available on the market contain at least one amide bond. One of the most sought after amides is dimethylformamide (DMF), which is used as an industrial solvent in the production of various textiles, plastics, chemicals, automotive, pulp and paper, electronics due to its low volatile, highly aprotic nature, and wide liquid-phase range. Currently, the one-step synthesis of DMF involves reacting N,N-dimethylamine (DMA) with carbon monoxide (CO), a toxic compound, in a sodium methoxide-methanol mixture at high pressures. The residual sodium methoxide-methanol mixture must be neutralized with acid or water. CO and inert off-gases are released by decompression from the high pressure, unreacted DMA and methanol are then separated by distillation to achieve high purity DMF. Alternatively, commercial grade methyl formate can be mixed with equimolar amounts of DMA. This results in a mixture of DMF and methanol than can further be separated and industrially used.

Given its desirability as a commodity chemical in subsequent reactions, efforts have been made to efficiently generate DMF and related carbonyl products. However, these methods generally require batch processing and stoichiometric oxidants, which necessitates the disposal of resultant by-products. Moreover, these methods also have issues regarding scalability, safety concerns associated with the use of toxic chemicals (e.g., carbon monoxide), and a high cost of reagents (e.g., methyl formate). Because of the low price and green routes to methanol, direct synthesis of DMF and other carbonyls from methanol and DMA would afford an improved synthesis technique without the need for dangerously high-pressure gases and expensive reagents. What are needed are new methods for preparing amides, like DMF having a high scalability and safer reagents. The methods and compositions disclosed herein address these and other needs.

### SUMMARY

Disclosed herein are compounds, compositions, methods for making and using such compounds and compositions. In various aspects disclosed herein are methods of forming an N,N-substituted-formamide comprising contacting a feed gas comprising an alcohol and an N,N-substituted-amine with a solid heterogeneous catalyst in the presence of an oxidizing agent at a reaction temperature, wherein the solid heterogeneous catalyst comprises one or more noble metals; and oxidatively coupling the feed gas under conditions effective to form an effluent gas comprising N,N-substituted-formamide.

In some aspects, the solid heterogeneous catalyst is disposed on a support, such as silica. In some aspects, the solid heterogenous catalyst comprises nanoparticles, and/or a nanoporous material.

In some aspects, the one or more noble metals are chosen from gold (Au), palladium (Pd), silver (Ag), platinum (Pt), copper (Cu), and combinations thereof. In some aspects, the solid heterogenous catalyst comprises palladium. In some aspects, the solid heterogenous catalyst comprises gold. In some aspects, the solid heterogenous catalyst comprises a bimetallic catalyst. In some aspects, the solid bimetallic catalyst comprises a Pd/Au alloy. In some aspects, the solid bimetallic catalyst has an atomic ratio of Pd to a second metal of from 100:1 to 1:1000, such as from 100:1 to 1:200, from 50:1 to 1:200, from 10:1 to 1:200, from 1:1 to 1:200, from 1:5 to 1:200, from 1:10 to 1:200, from 1:20 to 1:200, from 1:30 to 1:200, from 1:40 to 1:200, from 1:50 to 1:200, or from 1:50 to 1:100. In some aspects, the second metal comprises a noble metal, such as gold.

In some aspects, the N,N-substituted-amine comprises dimethylamine (DMA) and the N,N-substituted-formamide comprises dimethylformamide (DMF). In some aspects, the alcohol comprises methanol. In some aspects, the oxidizing agent comprises oxygen. In some aspects, the method is performed continuously or semi-continuously in a reactor, such as a packed-bed reactor. In some aspects, the catalytic selectivity of the N,N-substituted-formamide is 50% or more, such as 55% or more, 60% or more, 65% or more, 70% or more, 75% or more, 80% or more, 85% or more, 90% or more, 95% or more, 97% or more, 99% or more, or 100%. In some aspects, the reaction temperature is from 75° C. to 250° C., such as from 75° C. to 225° C., from 75° C. to 200° C., from 75° C. to 175° C., from 75° C. to 155° C., from 100° C. to 155° C., from 110° C. to 155° C., from 115° C. to 135° C., or 125° C.

In some aspects, the method further includes separating the N,N-substituted-formamide from the effluent gas, thereby forming a product gas stream comprising N,N-substituted-formamide and a residual gas. In some aspects, the N,N-substituted-formamide is separated from the product gas by distillation. In some aspects, the method further includes recirculating the residual gas to mix with the feed gas.

In some aspects, the molar ratio of the alcohol to the N,N-substituted-amine is 1:1 or more, such as 2:1 or more, 3:1 or more, 4:1 or more, 5:1 or more, 6:1 or more, 7:1 or more, 8:1 or more, 9:1 or more, 10:1 or more, 15:1 or more, 20:1 or more, or 25:1 or more. In some aspects, the selectivity of CO₂ is 20% or less, such as 15% or less, 10% or less, 5% or less, 4% or less, 3% or less, 2% or less, 1% or less, or 0%.

In another aspect, the present disclosure relates to method of forming a carbamide and/or a carbamate comprising contacting a feed gas comprising an alcohol and an N,N-substituted-amine with a solid heterogeneous catalyst in the presence of an oxidizing agent at a temperature greater than or equal to 155° C., wherein the solid heterogeneous catalyst comprises one or more noble metals; and oxidatively coupling the feed gas under conditions effective to form a product gas comprising the carbamide and/or carbamate.

In some aspects, the solid heterogeneous catalyst is disposed on a support, such as silica. In some aspects, the solid heterogenous catalyst comprises nanoparticles, and/or a nanoporous material. In some aspects, the one or more noble metals are chosen from gold (Au), palladium (Pd), silver (Ag), platinum (Pt), copper (Cu), and combinations thereof. In some aspects, the solid heterogenous catalyst comprises palladium. In some aspects, the solid heterogenous catalyst comprises gold. In some aspects, the solid heterogenous catalyst comprises a solid bimetallic catalyst. In some aspects, the solid bimetallic catalyst comprises a Pd/Au alloy. In some aspects, the solid bimetallic catalyst comprises an Ag/Au alloy.

In some aspects, the N,N-substituted-amine comprises dimethylamine (DMA). In some aspects, the alcohol comprises methanol. In some aspects, the oxidizing agent comprises oxygen. In some aspects, the carbamide comprises tetramethyl urea (TMU). In some aspects, the carbamide comprises imidazolida-2-one. In some aspects, the carbamate comprises methyldimethylcarbamate (MDMC). In some aspects, the temperature is from 155° C. to 250° C., such as from 155° C. to 225° C., from 155° C. to 200° C., or from 155° C. to 175° C.

Additional advantages of the disclosed subject matter will be set forth in part in the description that follows and the Figures, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIGS. 1A-1B show a comparison between the rate of DMF formation per gram of catalyst and carbon selectivity (FIG. 1A), and the rate per mol of dilute metal of five example catalysts (FIG. 1B).

FIG. 2 shows a reaction mechanism for the oxidative cross-coupling of methanol and dimethylamine on a dilute Ag in Au alloy surface.

FIG. 3 shows rate and selectivity data as a function of oxygen concentration over nanoporous gold.

FIG. 4 shows rate and selectivity data as a function of methanol concentration over nanoporous gold.

FIG. 5 depicts Arrhenius plots for DMF formation rates over npAu (diamonds), 1:15 AgAu/SiO₂ (triangles), Pd/SiO₂ (circles) and 1:10 PdAu/SiO₂ (squares). Reaction conditions: 143 kPa total pressure (2.85 kPa methanol, 1.4 kPa O₂, 0.086 kPa DMA, balance He), 373 K-448 K, npAu mass; 12.9 mg (GHSV: 170000 h⁻¹); 1:15 AgAu/SiO₂ mass; 6.7 mg (GHSV: 175000 h⁻¹); Pd/SiO₂ mass; 20.1 mg (GHSV: 20700 h⁻¹); 1:10 PdAu/SiO₂ mass; 5.4 mg (GHSV: 197000 h⁻¹); each diluted with Si-xerogel.

FIG. 6 shows reaction orders of DMA (circles), O₂ (triangles), and methanol (squares) on npAu. Reaction conditions: 143 kPa (2.9-5.9 kPa methanol, 1.4-4.3 kPa O₂, 0.019-0.094 kPa DMA, Balance He), 398K, npAu mass; 0.029 g (GHSV: 170000 h⁻¹′)

FIG. 7 shows results from a DMF co-feed experiment. Conditions: 143 kPa (2.9 kPa methanol, 1.4 kPa O₂, 0.065 kPa DMA, 0.0088 kPa DMF Balance He), 398K, npAu mass; 0.029 g.

FIG. 8 shows reaction orders of DMA (circles), O₂ (triangles), and methanol (squares) on PdAu/SiO₂. Reaction conditions: 141 kPa (1.00-5.70 kPa methanol, 1.10-8.56 kPa O₂, 0.014-0.156 kPa DMA, Balance He), 398 K, 1:10 PdAu/SiO₂ mass; 0.0054-0.0058 g diluted in Si-xerogel (approximately twice the catalyst mass) mg (GHSV: 197000 h⁻¹). Catalyst and Si-xerogel diluent sieved to 180 μm-250 μm.

FIG. 9 shows the DMF reaction order on 1:10 PdAu/SiO₂. Reaction conditions: 143 kPa (2.9 kPa methanol, 1.4 kPa O₂, 0.17-0.86 kPa DMA, 1.4 kPa CH 4, 0.00015-0.00084 kPa DMF, Balance He), 398 K, 1:10 PdAu/SiO₂ mass: 0.006 g diluted in Si-xerogel (approximately twice the catalyst mass) mg (GHSV: 197000 h⁻¹). Catalyst and Si-xerogel diluent sieved to 180 μm-250 μm.

FIG. 10 shows reaction orders of DMA (circles), O₂ (triangles), and methanol (squares) over Pd/SiO₂. Reaction conditions: 141 kPa (1.00-5.70 kPa methanol, 1.10-8.56 kPa O₂, 0.014-0.156 kPa DMA, Balance He), 398 K, Pd/SiO₂ mass; 0.0140-0.0201 g diluted in Si-xerogel (approximately equal the catalyst mass) mg (GHSV: 207000 h⁻¹). Catalyst and Si-xerogel diluent sieved to 180 μm-250 μm.

FIG. 11 shows Reaction orders of DMA (circles), O₂ (triangles; gray at lower 02 pressures and black at higher O₂ pressures on the same loading of catalyst weeks apart from one another), and methanol (squares). Reaction conditions: 141 kPa (2.85-14.27 kPa methanol, 0.72-28.54 kPa O₂, 0.019-0.156 kPa DMA, Balance He), 398 K, 1:15 AgAu/SiO₂ mass; 0.0093 g diluted in in Si-xerogel (approximately twice the catalyst mass) (GHSV: 175000 h⁻¹). Catalyst and Si-xerogel diluent sieved to 180 μm-250 μm.

FIG. 12 shows various SEM images of a PdAu bimetallic catalyst on a SiO₂ support.

FIG. 13 shows various SEM images of a AgAu bimetallic catalyst on a SiO₂ support.

FIG. 14 shows various TEM images of AgAu bimetallic catalyst disposed on a SiO₂ support and a bar graph showing variance in particle size distribution.

FIG. 15 shows various TEM images of PdAu bimetallic catalyst disposed on a SiO₂ support and a bar graph showing variance in particle size distribution.

FIGS. 16A-16B show DMF production rates (FIG. 16A) and selectivity (FIG. 16B) for a feed gas of methanol and DMA reacted over a heterogeneous catalyst (20 wt % Au/Si-xerogel-[Au/SiO₂]). Flowrate: 100 mL min⁻¹; Pressure: 135 kPa (2% Methanol, 1% O₂, 0.06% DMA, 0.11% CH₄, Balance He); Temperature: 398 K; Catalyst Mass: 0.0164 g; Conversion Methanol: 0%; DMA: 0%; O₂: 0% GHSV: 19100 hr⁻¹.

FIGS. 17A-17B show DMF production rates (FIG. 17A) and selectivity (FIG. 17B) for a feed gas of methanol and DMA reacted over a heterogeneous catalyst (1 wt % Pd/Si-xerogel-[Pd/SiO₂]). Flowrate: 100 mL min⁻¹; Pressure: 140 kPa (2% Methanol, 1%-20% O₂, 0.06% DMA, 0.11% CH₄, Balance He); Temperature: 398 K; Catalyst Mass: 0.014 g; Conversion Methanol: <8%; DMA: <10%; O₂: <5% GHSV: 20700 hr⁻¹.

FIGS. 18A-18B show DMF production rates (FIG. 18A) and selectivity (FIG. 18B) for a feed gas of methanol and DMA reacted over a heterogeneous catalyst (4.85 wt % (Pd+Au)/fumed silica—[1:10 Pd:Au/SiO₂]) Flowrate: 100 mL min⁻¹; Pressure: 142 kPa (0.6-3.3% Methanol, 1% O₂, 0.014% DMA, 1% CH₄, Balance He); Temperature: 398 K; Catalyst Mass: 0.0054 g; Methanol: <10%; DMA: <10%; O₂: <4%; GHSV: 197000 hr⁻¹.

FIGS. 19A-19B show DMF production rates (FIG. 19A) and selectivity (FIG. 19B) for a feed gas of methanol and DMA reacted over a heterogeneous catalyst (6.3 wt % (Pd+Au)/fumed silica—[1:2 PdAu/SiO₂]) Flowrate: 100 mL min⁻¹; Pressure: 142 kPa (2% Methanol, 1% O₂, 0.06% DMA, 1% CH₄, Balance He); Temperature: 398 K; Catalyst Mass: 0.0069 g; Conversion Methanol: <1%; DMA: <2%; O₂: <1% GHSV: 215000 hr⁻¹.

FIGS. 20A-20B show DMF production rates (FIG. 20A) and selectivity (FIG. 20B) for a feed gas of methanol and DMA reacted over a heterogeneous catalyst (4 wt % (Pd+Au)/fumed silica—[1:53 PdAu/SiO₂]). Flowrate: 100 mL min⁻¹; Pressure: 142 kPa (2% Methanol, 1% O₂, 0.06% DMA, 1% CH₄, Balance He); Temperature: 398 K; Catalyst Mass: 0.0069 g; Conversion Methanol: <1%; DMA: <10%; O₂: <1% GHSV: 214000 hr⁻¹.

FIGS. 21A-21B show DMF production rates (FIG. 21A) and selectivity (FIG. 21B) for a feed gas of methanol and DMA reacted over a heterogeneous catalyst (2 wt % (Ag+Au)/Si-xerogel—[1:15 AgAu/SiO₂]). Flowrate: 100 mL min⁻¹; Pressure: 142 kPa (2% Methanol, 1% O₂, 0.014-0.11% DMA, 0.11% CH₄, Balance He); Temperature: 398 K; Catalyst Mass: 0.0093 g; Conversion Methanol: <8%; DMA: <10%; O₂: <5% GHSV: 175000 h⁻¹.

FIG. 22 is a graph showing the effect of temperature on selectivity of certain products (DMF, TMU, IMD, and formamide) using PdAu/SiO₂ catalysts at ambient pressure.

FIG. 23 shows the formation of methyl dimethylcarbamate (MDMC) at elevated temperature (423K-473K) over a 1:15 AgAu/SiO₂ catalyst.

FIG. 24 shows the nitrogen selectivity of DMF and MDMC over a 1:15 AgAu/SiO₂ catalyst.

FIG. 25 shows SEM images of nanoporous gold.

FIGS. 26A-26D show N₂ isotherms of nanoporous gold (FIG. 26A), Pd/SiO₂ (FIG. 26B), 1:15 AgAu/SiO₂ (FIG. 26C), and 1:10 PdAu/SiO₂ (FIG. 26D).

FIG. 27 shows DMF production rates and selectivity for a feed gas of methanol and DMA reacted over a heterogeneous catalysts synthesized by strong electrostatic adsorption (SEA) methods. Flowrate: 100 mL min⁻¹; Pressure: 142 kPa (2% Methanol, 1% O₂, 0.07% DMA, 1.12% CH₄, Balance He); Temperature: 398 K; Catalyst Mass: 0.015-0.020 g; Conversion Methanol: <5%; DMA: <10%; O₂: <5% GHSV: 175000 hr⁻¹.

### DETAILED DESCRIPTION

The materials, compounds, compositions, articles, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

As used herein, the term “catalyst” refers to a substance the presence of which increases the rate and/or extent of a chemical reaction, while not being consumed as a reagent or undergoing a permanent chemical change itself. The term “heterogeneous catalyst” refers to a catalyst that is in a different phase (e.g., solid) than the phase of the reactants (e.g., gas) during catalysis. For example, a heterogeneous catalyst can include a catalyst having one or more metals (e.g., monometallic, bimetallic, trimetallic). As used herein, the term “bimetallic catalyst” refers to a catalyst having two metallic components.

As used herein, the terms “catalytic selectivity” and the like refer to a measure of a catalytic agent's ability to convert a reactant to a desired product relative to the amount of reactant converted. For example, the reaction can be selective to a compound such that 5% or more (e.g., 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, 99.9% or more, or 99.99% or more) of the reaction products comprise the compound.

“Phase,” as used herein, generally refers to a region of material having a substantially uniform composition which is a distinct and physically separate portion of a heterogeneous system. The term “phase” does not imply that the material making up a phase is a chemically pure substance, but merely that physical properties of the material making up the phase are essentially uniform throughout the material, and that these physical properties differ significantly from the physical properties of another phase within the material. Examples of physical properties include density, index of refraction, and chemical composition.

“Particle,” as used herein, generally refers to a discrete unit of material, such as a grain, bead, or other particulate form, typically with dimensions (length, width, and/or height) ranging from 1 μm to 100 μm. Particles may have any shape (e.g., spherical, ovoid, or cubic).

“Nanoparticle”, as used herein, generally refers to a particle of any shape having an average particle size from about 0.01 nm up to, but not including, about 1 micron. In certain aspects, nanoparticles have an average particle size from about 0.01 nm to 100 nm. The size of nanoparticles can be experimentally determined using a variety of methods known in the art, including electron microscopy.

“Mean particle size” or “average particle size”, are used interchangeably herein, and generally refer to the statistical mean particle size (diameter) of the nanoparticles in a population of nanoparticles. The diameter of an essentially spherical nanoparticle may refer to the physical or hydrodynamic diameter. The diameter of a non-spherical nanoparticle may refer preferentially to the hydrodynamic diameter. As used herein, the diameter of a non-spherical nanoparticle may refer to the largest linear distance between two points on the surface of the nanoparticle. Mean particle size can be measured using methods known in the art, such as evaluation by scanning electron microscopy.

“Monodisperse” and “homogeneous size distribution” are used interchangeably herein, and generally describe a population of nanoparticles where all of the nanoparticles are the same or nearly the same size. As used herein, a monodisperse distribution refers to particle distributions in which 90% of the distribution lies within 25% of the median particle size (e.g., within 20% of the median particle size, within 15% of the median particle size, within 10% of the median particle size, or within 5% of the median particle size).

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond.

Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O, which is also referred to herein as a “carbonyl.”

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “carbamide” means compounds represented by the form —N(Z¹)—(CO)N(Z¹)₂ where each Z¹ can be, independently, an alkyl, alkenyl, alkynyl, aryl, arylalkyl, cycloalkyl, carbonyl, ether, haloalkyl, heteroaryl and heterocyclyl.

The term “carbamate” means compounds represented by the form —Z¹OC(O)N(Z¹)—, —Z¹OC(O)N(Z¹) Z¹—, or —OC(O)N(Z¹) 2, where each Z¹ can be, independently, an alkoxy, aryloxy, alkyl, alkenyl, alkynyl, aryl, arylalkyl, cycloalkyl, ether, formyl, haloalkyl, heteroaryl, and heterocyclyl. Carbamates include, e.g., arylcarbamates and heteroaryl carbamates.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “formamide” refers to compounds comprising the —NC(O)H formamide group. Formamides include compounds having the formula HC(O)NZ¹Z² wherein Z¹ and Z² can be, independently, hydrogen or an alkyl, alkenyl, alkynyl, aryl, arylalkyl, cycloalkyl, carbonyl, ether, haloalkyl, heteroaryl and heterocyclyl.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Methods for Forming an N,N-Substituted-Formamide

Disclosed herein are methods for forming an N,N-substituted-formamide. In various aspects, the methods include contacting a feed gas comprising an alcohol and an N,N-substituted-amine with a solid heterogeneous catalyst in the presence of an oxidizing agent at a reaction temperature, wherein the solid heterogeneous catalyst comprises one or more noble metals; and oxidatively coupling the feed gas under conditions effective to form an effluent gas comprising N,N-substituted-formamide.

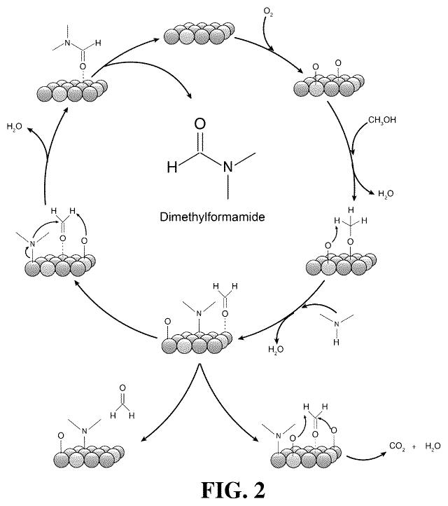

The present method is capable of forming various N,N-substituted-formamides, including, for example, N,N-dimethylformamide (DMF), N,N-diethylformamide, N-ethyl-N-methylformamide and N-methyl-N-phenylformamide. In various aspects the N,N-substituted-formamide comprises N,N-dimethylformamide (DMF). Without wishing to be bound by theory, the identity of the N,N-substituted-formamide product will largely depend on the N,N-substituted-amine reactant. An example reaction mechanism for the oxidative cross-coupling of methanol and dimethylamine on a dilute Ag in Au alloy surface is shown in FIG. 2. Non-limiting examples of suitable N,N-substituted-amines include, dimethylamine, diethylamine, dipropylamine, dibutylamine, dipentylamine, dihexylamine, diheptylamine, dioctylamine, dimethanolamine, diethanolamine, dipropanolamine, dibutanolamine, dipentanolamine, dihexanolamine, diheptanolamine, dioctanolamine, and mixtures thereof. In some aspects, the N,N-substituted-amine comprises dimethylamine.

In some examples, the N,N-substituted-amine has a normal boiling point of 250° C. or less, such as 225° C. or less, 200° C. or less, 175° C. or less, 150° C. or less, 140° C. or less, 130° C. or less, or 120° C. or less. In some examples, the N,N-substituted-amine has a normal boiling point of 50° C. or more, such as 60° C. or more, 75° C. or more, 100° C. or more, 125° C. or more, 150° C. or more, 175° C. or more, or 200° C. or more. In some aspects, the N,N-substituted-amine has a normal boiling point ranging from any of the lower values to any of the higher values (e.g., from 50° C. to 250° C., from 50° C. to 225° C., from 50° C. to 200° C., from 50° C. to 175° C., from 50° C. to 150° C., from 60° C. to 150° C., from 75° C. to 150° C., or from 100° C. to 150° C.). Boiling points are at ambient pressure.

The feed gas further includes an alcohol. In various aspects, the alcohol has a normal boiling point of 250° C. or less, such as 225° C. or less, 200° C. or less, 175° C. or less, 150° C. or less, 140° C. or less, 130° C. or less, or 120° C. or less. In some examples, the alcohol has a normal boiling point of 50° C. or more, such as 60° C. or more, 75° C. or more, 100° C. or more, 125° C. or more, 150° C. or more, 175° C. or more, or 200° C. or more. In some aspects, the alcohol has a normal boiling point ranging from any of the lower values to any of the higher values (e.g., from 50° C. to 250° C., from 50° C. to 225° C., from 50° C. to 200° C., from 50° C. to 175° C., from 50° C. to 150° C., from 60° C. to 150° C., from 75° C. to 150° C., or from 100° C. to 150° C.). Boiling points are at ambient pressure.

Some exemplary alcohols for the present methods include methanol, ethanol, propanol, butanol, hexanol, heptanol, octanol, nonanol, decanol, benzyl alcohol, iso-butyl alcohol, n-butyl alcohol, 2-ethyl hexanol, furfuryl alcohol, iso-propyl alcohol, or n-propyl alcohol. In various aspects, the alcohol comprises methanol. In some examples the alcohol can be ethanol, propanol, and/or butanol. An oxidizing agent is additionally used in the catalysis of the N,N-substituted-amine and alcohol. Examples of gaseous oxidizing agents are steam, carbon dioxide, and gases containing molecular oxygen, (e.g., air, pure molecular oxygen, or molecular oxygen diluted with helium, argon, nitrogen, or other non-oxidizing gases).

As used herein, the term “noble metals” refers to the elements in Groups 10 and 11 of the periodic table of elements. The heterogeneous catalyst discussed herein can include one or more noble metals. For example, the solid heterogeneous catalyst can be a bimetallic catalyst comprising an alloy of one or more noble metals (e.g., Pd/Au, Pd/Ag, Ag/Au) or a bimetallic catalyst of a noble metal and a base metal. The term “base metals” refers to metals not in either of Groups 10 or 11, such as iron, zinc, aluminum, tin, tungsten, molybdenum, tantalum, cobalt, bismuth, titanium, zirconium, antimony, manganese, beryllium, germanium, vanadium, gadolinium, hafnium, indium, niobium, rhenium, cerium, lanthanum, praseodymium, neodymium, and a combination thereof. In some aspects, the one or more noble metals include, for example, palladium, silver, platinum, gold, copper, and combinations thereof. In various aspects, the solid heterogenous catalyst comprises palladium. In some aspects, the solid heterogenous catalyst comprises gold.

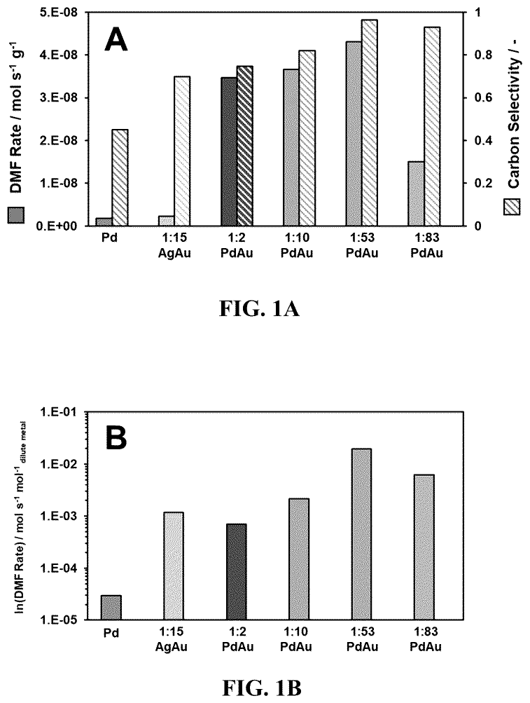

In various aspects, the heterogeneous catalyst includes a bimetallic catalyst having an atomic ratio of a first metal (e.g., Pd) to a second metal (e.g., Au). For example, the atomic ratio of the first metal can range from 100:1 to 1:1000, such as from 50:1 to 1:1000, from 10:1 to 1:200, from 1:1 to 1:200, from 1:5 to 1:200, from 1:10 to 1:200, from 1:20 to 1:200, from 1:30 to 1:200, from 1:40 to 1:200, from 1:50 to 1:200, or from 1:50 to 1:1000. As shown in FIGS. 1A-1B, a bimetallic catalyst of 1:50 Pd/Au was surprisingly shown to exhibit higher DMF rate and selectivity compared to catalysts having a larger atomic ratio of Pd.

The heterogeneous catalyst can be disposed on a support to impart stability, to increase effective surface area, or to influence other properties of the heterogeneous catalyst. For example, the catalyst can be dispersed on a support using various synthesis methods, including but not limited to strong electrostatic adsorption (SEA), dry impregnation or incipient wetness impregnation (IWI), charge enhanced dry impregnation (CEDI), precipitation, reactive deposition, coprecipitation, deposition precipitation, precipitation-impregnation. For example, strong electrostatic adsorption (SEA) can be used and generally involves controlling the pH of a metal precursor solution relative to the point of zero charge of the support. Thus, the electrostatic interaction between the charged surface groups (typically hydroxyl groups) and the oppositely charged metal coordination complex can be exploited, creating a strong metal attachment at a high loading while also maintaining a high amount of metal dispersion within the support.

In various aspects, the support comprises carbon, activated carbon, graphite, silica, titania, alumina, calcium silicate, calcium carbonate, silica-alumina, silica aluminate, zirconia, barium carbonate and barium sulfate. In some aspects, the support comprises silica. The heterogeneous catalyst disposed on the support can further form nanoparticles, having, for example, a size distribution ranging from 0.01 nm to 100 nm. In some examples, the nanoparticles can have an average particle size of 100 nm or less, 90 nm or less, 80 nm or less, 70 nm or less, 60 nm or less, 50 nm or less, 40 nm or less, 30 nm or less, 20 nm or less, or 10 nm or less. In further examples, the nanoparticles can have an average particle size of from 100 nm to 90 nm, 100 nm to 80 nm, 100 nm to 70 nm, 100 nm to 60 nm, 100 nm to 50 nm, 100 nm to 40 nm, 100 nm to 30 nm, 100 nm to 20 nm, 100 nm to 10 nm, or 100 nm to 0.01 nm. In certain examples, the nanoparticles can have an average particle size of from 0.01 nm to 10 nm, 0.01 nm to 20 nm, 0.01 nm to 30 nm, 0.01 nm to 40 nm, 0.01 nm to 50 nm, 0.01 nm to 60 nm, 0.01 nm to 70 nm, 0.01 nm to 80 nm, 0.01 nm to 90 nm, or 0.01 nm to 100 nm. In specific examples, the nanoparticles can have an average particle size of from 0.01 nm to 10 nm, 10 nm to 20 nm, 20 nm to 30 nm, 30 nm to 40 nm, 40 nm to 50 nm, 50 nm to 60 nm, 60 nm to 70 nm, 70 nm to 80 nm, 80 nm to 90 nm, or 90 nm to 100 nm. In some examples, the nanoparticles can have an average particle size of from 0.01 nm to 25 nm, 25 nm to 50 nm, 50 nm to 75 nm, or 75 nm to 100 nm. In some aspects, the nanoparticles are substantially monodisperse.

In some aspects, the heterogeneous catalyst forms a nanoporous material. As used herein, the term “nanoporous material” refers to a porous material having an average pore size in the range of 1 nm to 100 nm. The nanoporous material can include a plurality of interconnected tunnels or “nanopores.” The pores can interconnect, resulting in a network of pores or voids that spans the material, permitting the flow of liquid or gas into and through the material, i.e., a continuous phase of pores or voids. The nanoporous material can comprise a substantially pure material (e.g., pure gold) or a combination of materials (e.g., Au/Ag, Pd/Au alloys). A nanoporous material can be provided using any technique known in the art. For example, the nanoporous material can be obtained using a dealloying process (e.g., free-corrosion approaches and/or via electrochemical processing), which refers to the selective dissolution of an alloy component under conditions where the remaining component diffuses along the alloy/solution interface to re-form into a highly porous metal (Erlebacher, J., Seshardi, R. Hard Materials with Tunable Porosity. MRS Bulletin 34, 561-566 (2009)).

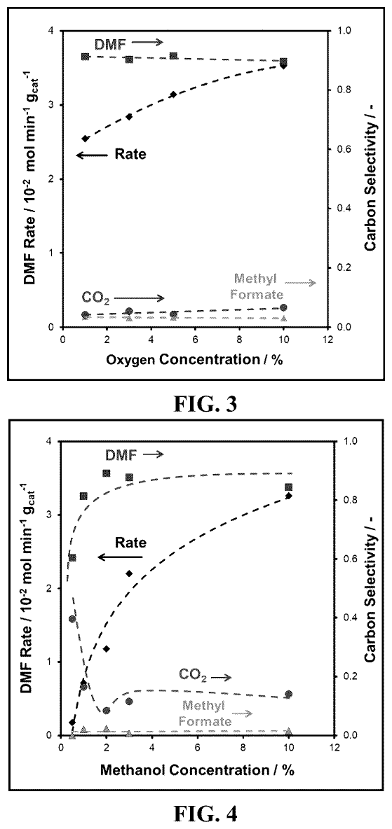

The present methods are able to selectively produce an N,N-substituted-formamide product. In some aspects, the catalytic selectivity of the N,N-substituted-formamide is 50% or more, such as 55% or more, 60% or more, 65% or more, 70% or more, 75% or more, 80% or more, 85% or more, 90% or more, 95% or more, 97% or more, 99% or more, or 100%. In some aspects, the selectivity of CO 2 is 20% or less, such as 15% or less, 10% or less, 5% or less, 4% or less, 3% or less, 2% or less, 1% or less, or 0%. FIGS. 3 and 4 show selectivity data of N,N-dimethylformamide (DMF), CO₂, and methyl formate for varying oxygen and methanol concentrations.

The present disclosure can additionally be performed continuously or semi-continuously (e.g., in a packed bed reactor). During continuous operation, the effluent stream comprising a mixture of the unreacted reagents and N,N-substituted-formamide product can be separated into a product stream substantially comprising the N,N-substituted-formamide and a residual gas stream. For example, a distillation process can be used to separate the N,N-substituted-formamide from the unreacted reagents. Other gas separation techniques are known in the art and can be incorporated without undue experimentation. The residual stream can further be recirculated to the feed gas, where it goes through oxidative coupling to increase the conversion of the reagents.

The term “feed gas” or “feed gases” refers to reactive gas(es) used in the processes according to the present disclosure. The terms “effluent gas” and “product gas” are used interchangeably and refer to a gas produced as a waste product, byproduct, or intended product from a reaction (e.g., a catalysis reaction).

As used herein, “conditions effective” refers to conditions to which a feed gas is subjected such that the feed gas is sufficiently converted into a product containing effluent stream. Conditions can include reaction temperature, pressure, reaction time, and the like. In various aspects of the present method, the reaction temperature is from 75° C. to 250° C., such as from 75° C. to 225° C., from 75° C. to 200° C., from 75° C. to 175° C., from 75° C. to 155° C., from 100° C. to 155° C., from 110° C. to 155° C., from 115° C. to 135° C., or 125° C. Temperatures within this range are shown to increase selectivity towards a N,N-substituted-formamide product. In some aspects, the molar ratio of the alcohol to the N,N-substituted-amine in the feed gas is 1:1 or more, such as 2:1 or more, 3:1 or more, 4:1 or more, 5:1 or more, 6:1 or more, 7:1 or more, 8:1 or more, 9:1 or more, 10:1 or more, 15:1 or more, 20:1 or more, or 25:1 or more.

### Methods for Forming a Carbamide/Carbamate

Disclosed herein are methods of forming a carbamide and/or a carbamate. In various aspects, the methods include contacting a feed gas comprising an alcohol and an N,N-substituted-amine with a solid heterogeneous catalyst in the presence of an oxidizing agent at a temperature greater than or equal to 155° C., wherein the solid heterogeneous catalyst comprises one or more noble metals; and oxidatively coupling the feed gas under conditions effective to form a product gas comprising the carbamide and/or carbamate.

The present methods are capable of forming various carbamide and/or carbamate products. Exemplary carbamides include imidazolida-2-one, N,N′-dimethylethylene urea, N,N′-diethylethylene urea, and N,N′-dimethylpropylene urea as well as, primarily, open-chained compounds such as N,N,N′,N′-tetramethyl urea (TMU) and N,N,N′,N′-tetraethyl urea. Exemplary carbamates include methyldimethylcarbamate (MDMC), butyl N,N-dimethyl carbamate and butyl N,N-diethyl carbamate as well as, primarily, cyclic esters such as 3-methyl-2-oxazolidinone, 3-ethyl-2-oxazolidinone, 3-propyl-2-oxazolidinone, 3-butyl-2-oxazolidinone, 3-pentyl-2-oxazolidinone, 3,4-dimethyl-2-oxazolidinone, 3,5-dimethyl-2-oxazolidinone, 3-methyl-4-ethyl-2-oxazolidinone, and 3-ethyl-5-methyl-2oxazolidinone.

In some examples, the N,N-substituted-amine has a normal boiling point of 250° C. or less, such as 225° C. or less, 200° C. or less, 175° C. or less, 150° C. or less, 140° C. or less, 130° C. or less, or 120° C. or less. In some examples, the N,N-substituted-amine has a normal boiling point of 50° C. or more, such as 60° C. or more, 75° C. or more, 100° C. or more, 125° C. or more, 150° C. or more, 175° C. or more, or 200° C. or more. In some aspects, the N,N-substituted-amine has a normal boiling point ranging from any of the lower values to any of the higher values (e.g., from 50° C. to 250° C., from 50° C. to 225° C., from 50° C. to 200° C., from 50° C. to 175° C., from 50° C. to 150° C., from 60° C. to 150° C., from 75° C. to 150° C., or from 100° C. to 150° C.). Boiling points are at ambient pressure.

Suitable N,N-substituted-amines include, dimethylamine, diethylamine, dipropylamine, dibutylamine, dipentylamine, dihexylamine, diheptylamine, dioctylamine, dimethanolamine, diethanolamine, dipropanolamine, dibutanolamine, dipentanolamine, dihexanolamine, diheptanolamine, dioctanolamine, and mixtures thereof. In some aspects, the N,N-substituted-amine comprises dimethylamine.

The feed gas further includes an alcohol. In various aspects, the alcohol has a normal boiling point of 250° C. or less, such as 225° C. or less, 200° C. or less, 175° C. or less, 150° C. or less, 140° C. or less, 130° C. or less, or 120° C. or less. In some examples, the alcohol has a normal boiling point of 50° C. or more, such as 60° C. or more, 75° C. or more, 100° C. or more, 125° C. or more, 150° C. or more, 175° C. or more, or 200° C. or more. Boiling points are at ambient pressure.

Some exemplary alcohols for the present methods include methanol, ethanol, propanol, butanol, hexanol, heptanol, octanol, nonanol, decanol, benzyl alcohol, iso-butyl alcohol, n-butyl alcohol, 2-ethyl hexanol, furfuryl alcohol, iso-propyl alcohol, or n-propyl alcohol. In various aspects, the alcohol comprises methanol. An oxidizing agent is additionally used in the catalysis of the N,N-substituted-amine and alcohol. Examples of gaseous oxidizing agents are steam, carbon dioxide, and gases containing molecular oxygen, (e.g., air, pure molecular oxygen, or molecular oxygen diluted with helium, argon, nitrogen, or other non-oxidizing gases).

As explained above, the term “noble metals” refers to the elements in Groups 10 and 11 of the periodic table of elements. The heterogeneous catalyst discussed herein includes one or more noble metals. For example, the solid heterogeneous catalyst can be a bimetallic catalyst comprising an alloy of one or more noble metals (e.g., Pd/Au, Pd/Ag, Ag/Au) or a bimetallic catalyst of a noble metal and a base metal. Exemplary base metals include iron, zinc, aluminum, tin, tungsten, molybdenum, tantalum, cobalt, bismuth, titanium, zirconium, antimony, manganese, beryllium, germanium, vanadium, gadolinium, hafnium, indium, niobium, rhenium, cerium, lanthanum, praseodymium, neodymium, and a combination thereof. In some aspects, the one or more noble metals include, for example, palladium, silver, platinum, gold, copper, and combinations thereof. In various aspects, the solid heterogenous catalyst comprises palladium. In some aspects, the solid heterogenous catalyst comprises gold.

The heterogeneous catalyst can also be disposed on a support to impart stability, to increase effective surface area, or to influence other properties of the catalyst. In some examples, the catalyst is dispersed on the support using strong electrostatic adsorption (SEA), dry impregnation or incipient wetness impregnation (IWI), charge enhanced dry impregnation (CEDI), precipitation, reactive deposition, coprecipitation, deposition precipitation, and/or precipitation-impregnation. In various aspects, the support comprises carbon, activated carbon, graphite, silica, titania, alumina, calcium silicate, calcium carbonate, silica-alumina, silica aluminate, zirconia, barium carbonate and barium sulfate. In some aspects, the support comprises silica.

The heterogeneous catalyst disposed on the support can further form nanoparticles, having, for example, a size distribution ranging from 0.01 nm to 100 nm. In some examples, the nanoparticles can have an average particle size of 100 nm or less, 90 nm or less, 80 nm or less, 70 nm or less, 60 nm or less, 50 nm or less, 40 nm or less, 30 nm or less, 20 nm or less, or 10 nm or less. In further examples, the nanoparticles can have an average particle size of from 100 nm to 90 nm, 100 nm to 80 nm, 100 nm to 70 nm, 100 nm to 60 nm, 100 nm to 50 nm, 100 nm to 40 nm, 100 nm to 30 nm, 100 nm to 20 nm, 100 nm to 10 nm, or 100 nm to 0.01 nm. In certain examples, the nanoparticles can have an average particle size of from 0.01 nm to 10 nm, 0.01 nm to 20 nm, 0.01 nm to 30 nm, 0.01 nm to 40 nm, 0.01 nm to 50 nm, 0.01 nm to 60 nm, 0.01 nm to 70 nm, 0.01 nm to 80 nm, 0.01 nm to 90 nm, or 0.01 nm to 100 nm. In specific examples, the nanoparticles can have an average particle size of from 0.01 nm to 10 nm, 10 nm to 20 nm, 20 nm to 30 nm, 30 nm to 40 nm, 40 nm to 50 nm, 50 nm to 60 nm, 60 nm to 70 nm, 70 nm to 80 nm, 80 nm to 90 nm, or 90 nm to 100 nm. In some examples. the nanoparticles can have an average particle size of from 0.01 nm to 25 nm, 25 nm to 50 nm, 50 nm to 75 nm, or 75 nm to 100 nm.

In some aspects, the heterogeneous catalyst forms a nanoporous material. As discussed above, the term nanoporous material refers to a porous material having an average pore size in the range of 1 nm to 100 nm. The nanoporous material can include a plurality of interconnected tunnels or “nanopores.” The nanoporous material can include a substantially pure material (e.g., pure gold) or a combination of materials (e.g., Au/Ag, Pd/Au alloys). In various aspects, the heterogeneous catalyst includes a bimetallic catalyst having an atomic ratio of a first metal (e.g., Pd) to a second metal (e.g., Au). For example, the atomic ratio of the first metal can range from 100:1 to 1:200, such as from 50:1 to 1:200, from 10:1 to 1:200, from 1:1 to 1:200, from 1:5 to 1:200, from 1:10 to 1:200, from 1:20 to 1:200, from 1:30 to 1:200, from 1:40 to 1:200, from 1:50 to 1:200, or from 1:50 to 1:100. The present disclosure found that higher temperatures can promote the formation of carbamide and carbamate products. Thus, in some aspects, the catalysis temperature can be from 155° C. to 250° C., such as from 155° C. to 225° C., from 155° C. to 200° C., or from 155° C. to 175° C.

The methods described herein can occur in any suitable reactor. For example, the processes could utilize a series of fixed bed reactors, where each reactor could be independently regenerated, a moving bed reactor where the catalysts moves through the reactor and is regenerated in a separate section of the plant, or a fluid bed reactor, where the catalyst is circulated through the reactor and regenerated in a separate vessel.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Catalyst Synthesis:

### Dealloyed White Gold

Synthesis of nanoporous gold was completed following free-corrosion procedures similar to those used previously. White gold leaves (70% Ag, 30% Au, W&B Gold Leaf, LLC) were place into a clean 120 cm³ perfluoroalkoxy (PFA) jar (Savillex Corp.) and 70% nitric acid (Sigma Aldrich, 70%) were added drop-wise to the jar to dissolve the leaves. The solution was stirred between 1800 s and 48 h to create samples with varied Ag content. The solids were centrifuged (5000 RPM) and washed with deionized water three times with deionized water and then dried in a drying oven at 363 K in stagnant air for 24 h.

Samples were heated to reaction temperature (0.033 K s⁻¹) under 1.67 cm³ g_{cat}⁻¹ s⁻¹ of 10% O₂ in balance He.

Ag, Au, and AgAu/SiO₂

### Synthesis of Ag—Au Bimetallic Catalysts Followed the Procedures of Liu et al.

SiO₂ was functionalized with 3-aminopropyltriethoxysilane (Sigma Aldrich, 99%) by ethanol reflux for 24 h. The solids were washed with ethanol (VWR, 99.9%) five times. and dried before being dispersed in ambient temperature Millipore H₂O (18.2 MΩ). A 1.65 wt % HAuCl₄ solution (VWR, Purity) was added while stirring to introduce Au ions. After more filtration and washing the solids were reduced by stirring in 0.2 M NaBH 4 (Sigma Aldrich, >98.0%). The samples were washed with Millipore H₂O five times to remove Cl ions before the Ag was added to the solution. AgNO₃ (Sigma Aldrich, 99%) was used as a precursor and was added in different ratios to create a molar ratio of 1:1 Ag:Au, although other ratios (e.g., 1:2, 1:3, 1:4, 1:5, 1:10, 1:25, 1:50, 1:100, 1:200) can also be used. The solid was calcined in flowing air (1.67 cm³ g_{cat}⁻¹ s⁻¹) at 723 K for 6 hours and then in flowing H₂ (Airgas, UHP, 1.67 cm³ g_{cat}⁻¹ s⁻¹) at 823 K for 1 hour to obtain the final Ag—Au/SiO₂. Ag/SiO₂ was synthesized with procedures also for Liu et al. without the Au galvanic replacement step.

PdAu/SiO₂ catalysts were synthesized following the procedure from Liu et al. HAuCl₄·3H₂O (VWR, 99.999%) was mixed with polyvinylpyrrolidone (PVP, Sigma Aldrich, Lot #WXBD4555V) and ethylene glycol (VWR, >99%) in a 500 cm³ round bottom flask. The solution was stirred and purged for one hour with N₂ (Airgas, UHP, 0.83 cm³ s⁻¹) before NaHCO₃ (VWR, >99.7%) was added. The solution was then heated in an oil bath to 363 K for 1800 s under the N₂ protection until unsupported Au NPs were formed in solution. Once cooled to ambient temperature, Pd(NO₃)₂ (Sigma Aldrich, 99%) is mixed with ethylene glycol and injected into the solution with a syringe. 150 cm³ of acetone (VWR, 99.5%) was added by syringe through a septa sealing a neck of the round bottom flask in order to precipitate the NPs. The solution was centrifuged (10000 RPM) to separate the solids from the acetone and ethylene glycol. The solids are washed with ethanol (VWR, 99.9%) and hexane (VWR, 99.5%) and dried in a vacuum oven (762 Torr of vacuum) at 298 K. Fumed silica (CABOT, Lot #4829189) was introduced into an Au NPs and H₂O solution and stirred overnight. The solids were separated by centrifugation and dried in a vacuum at ambient temperature for 24 h.

Bimetallic PdAu/SiO₂ catalysts were synthesized by strong electrostatic adsorption methods described by Dong et al. First, 100 ppm solutions of Pd(NH₃)₄ and Au bis-ethylenediamine are created by diluting 10 wt % Pd(NH₃)₄ in H₂O (Sigma, >99.99%) with millipore H₂O (18.2 MΩ) and by dissolving in-house synthesized Au bis-ethylenediamine. The volume of precursor solution used for each synthesized was determined by the surface area and quantity of the support material (1000 m² L⁻¹). Typically, the surface area of Si-Xerogel was assumed to be 200 m² g⁻¹. The precursor solutions were mixed together, and the pH of the solution was measured with Mettler Toledo FiveEasy Plus pH meter. The pH of the solution was adjusted with 4M NH 3 OH to 10.5. Then, 0.9 g of finely ground Si-Xerogel was mixed into the precursor solution. The pH was again adjusted to 10.5 with 4M NH 4 OH. The mixture was left to stir for 1 hour. The powder was reclaimed by centrifugation and left to dry in dark, ambient conditions (room temperature and atmospheric pressure) for 4-5 days. Once dry, the powders were reduced in a tube furnace at 673K for 1 hour under 20% H₂ in He (1 K min⁻¹ ramp rate, total flow of 100 sccm g⁻¹ of powder).

Pd/SiO₂ was prepared with an incipient wetness procedure. 0.2 g of SiO₂ was stirred in 1 mL of H₂O. Then, 0.1756 g of Pd(NO₃)₂ was mixed with water and added dropwise into the silica slurry.

PdCu/SiO₂ catalysts were synthesized with procedures from Shan et al. Cu/SiO₂ were synthesized under nitrogen protection (0.83 cm³ s⁻¹ flow) in a 500 cm³ round bottom flask with a mixture of Cu(NO₃)₂ (Sigma, 99.99%) and PVP in 0.1 M ascorbic acid (VWR, 99%). NaBH₄ (0.1 M) was added dropwise until the solution turned brown. Fumed silica was activated by treatment in flowing air at 923 K for 12 h in a muffle furnace (0.0167 K s⁻¹ ramp rate). The fumed silica was then suspended in H₂O and added drop-wise to the round bottom flask. The mixture was stirred under N₂ protection for 1800 s and then centrifuged and washed with deionized water several times. The collected solids were dried in a vacuum oven (762 Torr of vacuum) at ambient temperature for 12 h and calcined to 623 K for 4 h and subsequently treated in a flow of hydrogen (Airgas, UHP, 1.67 cm³ g_{cat}⁻¹ s⁻¹) for 3 h to obtain silica supported Cu NPs. Pd atoms were deposited onto the Cu surface by galvanic replacement. The Cu/SiO₂ were suspended in ambient temperature water and Pd(NO 3) 6H₂O was added. Then, under N₂ protection (flow of 0.83 cm³ s⁻¹), the solution was mixed for 1 h. The solids were filtered and washed with deionized water again. The final solids were dried in vacuum at 333 K and treated in a flow of 10% hydrogen at 623 K for 1 h.

### Catalyst Characterization

### N₂ Adsorption—Micropore Volumes

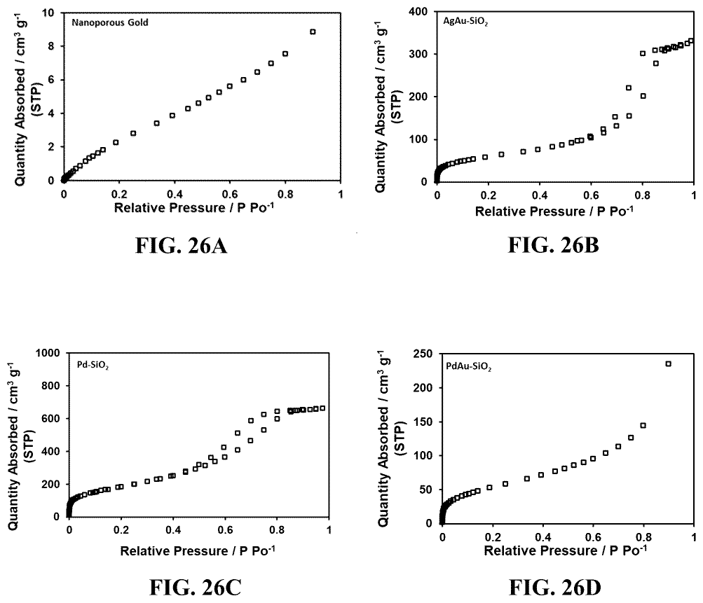

N₂ physisorption was performed using a Micrometrics ASAP2020 Plus. N₂ isotherms of various catalysts including nanoporous gold, Pd/SiO₂, 1:15 AgAu/SiO₂, and 1:10 PdAu/SiO₂ are depicted in FIGS. 26A-26D. Samples were pre-treated then pelleted using a pellet press (Carver) and die set (Specac) at 8000 psi, and ground to 180-250 μm, before loading in a glass vacuum tube for degassing at 623 K at less than 1 μmHg for 9 h.

Then, the sample tubes were re-weighed, the degassed sample mass calculated, and the tube loaded to the analysis port for N₂ adsorption (77 K).

TABLE 1
Catalyst characterization results (ICP, N₂ adsorption,
XRD, TEM) for the samples tested.

| | Total Metal | | BET | XRD Average | TEM Average |
|---|---|---|---|---|---|
| | Weight | Metal | Surface | Particle | Particle |
| Catalyst | Loading | Molar | Area | Size | Size |
| Composition | (wt %) | Ratio | (m²/g) | (nm)^{c} | (nm)^{d} |
| Au/SiO₂ | 20 ^{b} | — | — | — | — |
| Pd/SiO₂ | 0.6 ^{b} | — | 563 ± 9 | — | 13 |
| NiCu/SiO₂ | 8 ^{b} | 1:100 ^{b} | 152 ± 3 | — | — |
| PdCu/SiO₂ | 8 ^{b} | 1:50 ^{b} | 148 ± 4 | — | 3 |
| AgAu/SiO₂ | 3.5 ^{b} | 1:15 ^{b} | 182 ± 3 | 15 | 15 |
| NiAu/SiO₂ | 5 | 1:10 | 86 ± 3 | — | 13 |
| PtAu/SiO₂ | 4 | 1:50 | 73 ± 3 | — | 3 |
| PdAu/SiO₂ | 2.5 ^{a} | 1:2 ^{a} | 180 ± 3 | 78 | 28 |
| PdAu/SiO₂ | 3.5 ^{a} | 1:10 ^{a} | 158 ± 3 | 23 | 15 |
| PdAu/SiO₂ | 2.3 ^{b} | 1:53 ^{b} | 173 ± 4 | 48 | 13 |
| PdAu/SiO₂ | 4 ^{b} | 1:83 ^{b} | 140 ± 3 | — | 14 |

^{a} Weight loadings and molar ratios measured by ICP-OES.
^{b} Weight loadings and molar ratios based on synthesis protocol.
^{c}Determined by Scherrer's equation using most prominent powder XRD peak.
^{d}Determined from size distribution of at least 75 particles counted per sample.

### Elemental Analysis

Inductively coupled plasma optical emission spectroscopy (ICP-OES) was used to determine the bulk elemental compositions of the various materials. Samples were digested in hydrofluoric acid (HF, 48%, VWR, 48%) overnight, followed by further digestion in aqua regia overnight, then dilution in nitric acid (prepared from 70% nitric acid) in PTFE, jars. The instrument was calibrated before each use using standards prepared from 1000 ppm standards for Ag, Au, Cu, and Pd.

### Kinetic Studies

Kinetic studies were conducted using as a continuous flow reactor. The effluent stream of the reactor was analyzed using an Agilent 6890N/5975 GC/MS equipped with a TCD and FID. Columns used for chemical separation are Alltech AT-Q and a Restek Shincarbon ST in series to separate permanent gases preceding the TCD and an Agilent Porabond U for hydrocarbon separation on the FID.

### DISCUSSION

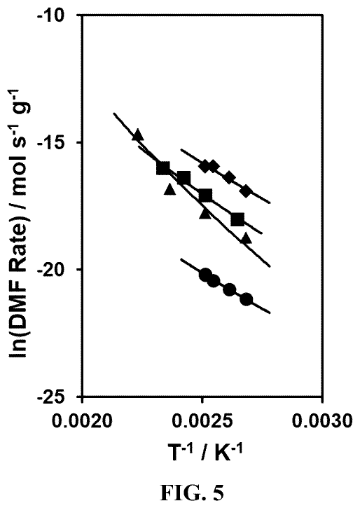

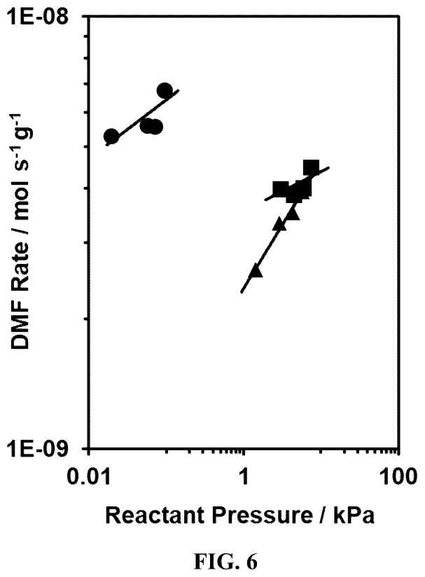

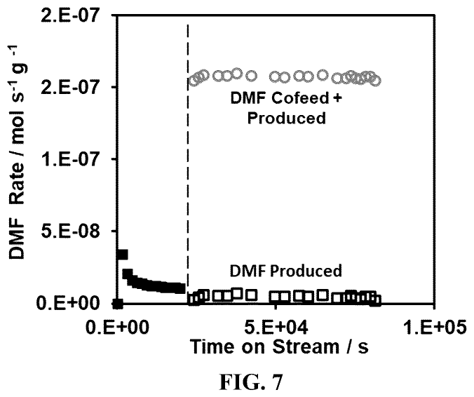

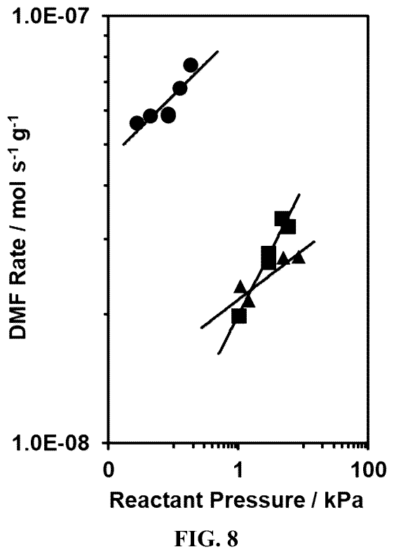

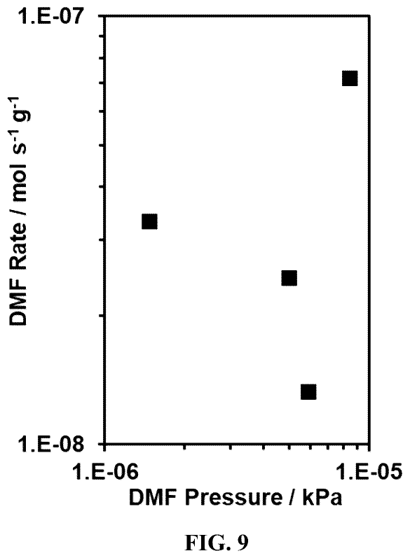

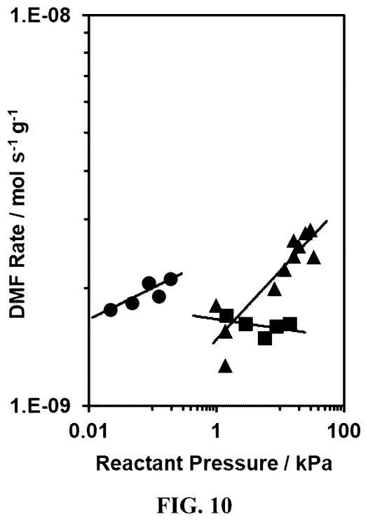

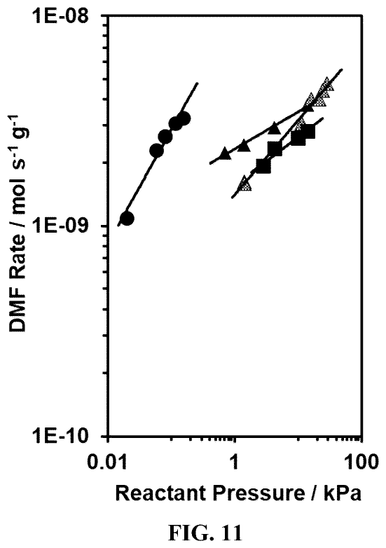

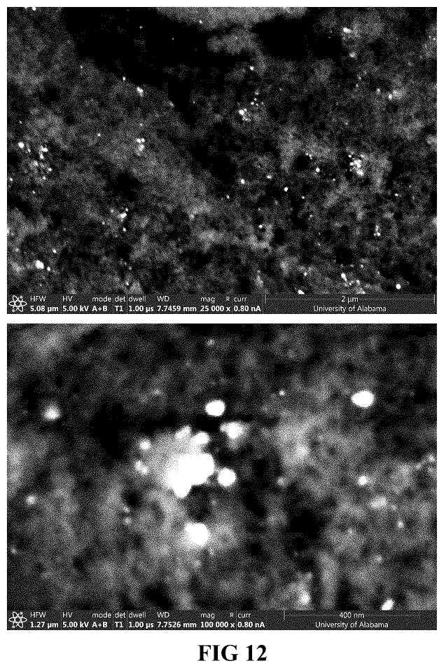

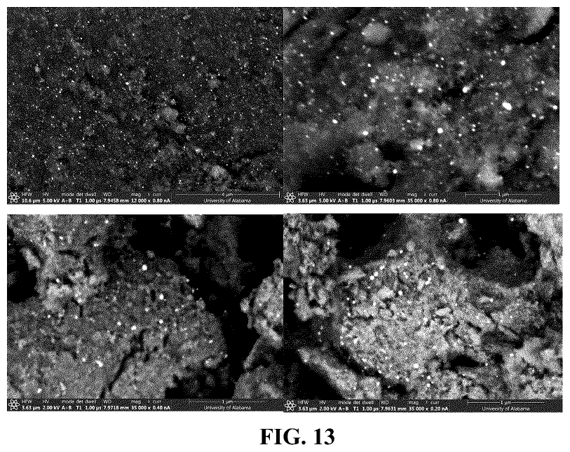

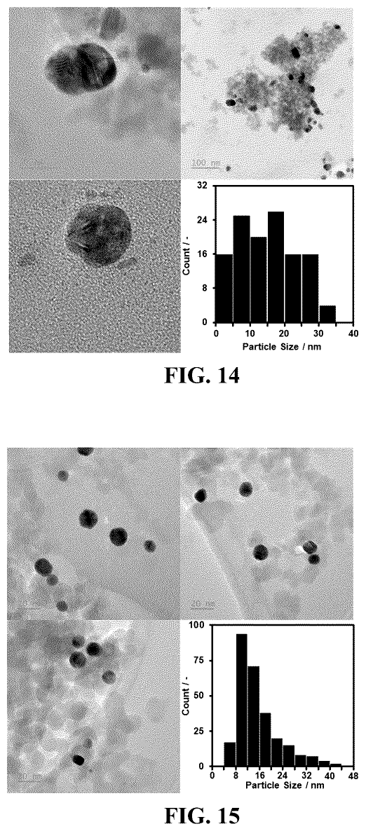

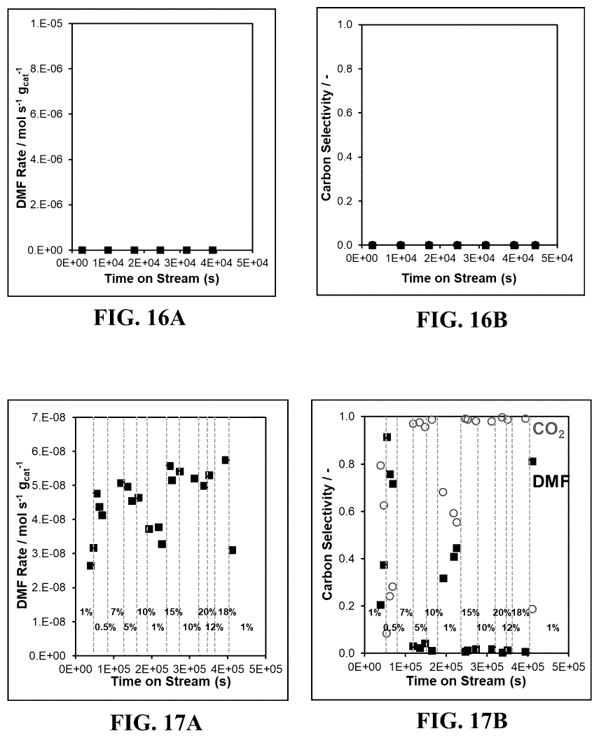

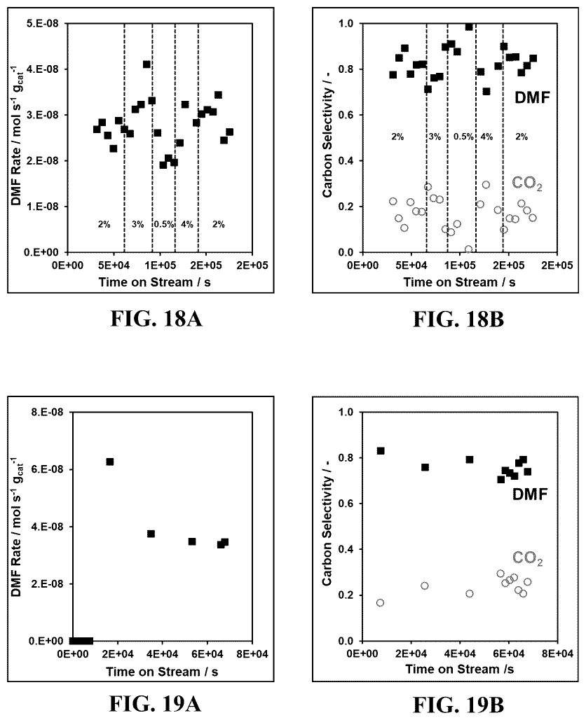

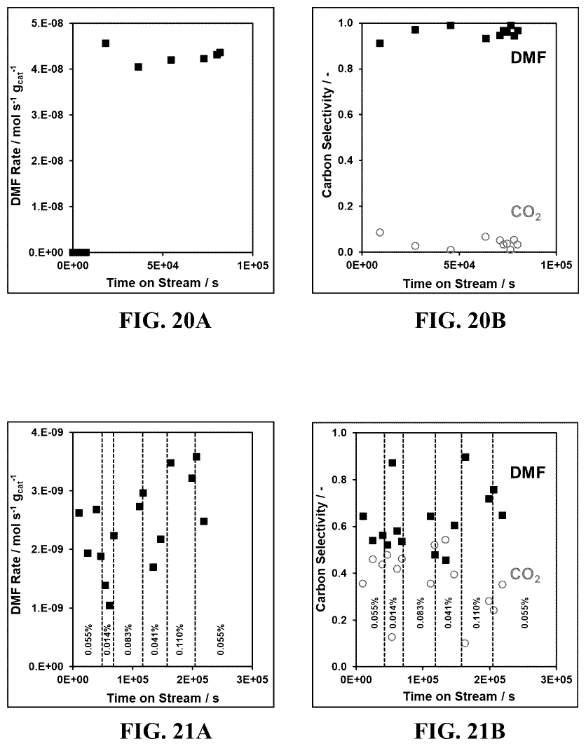

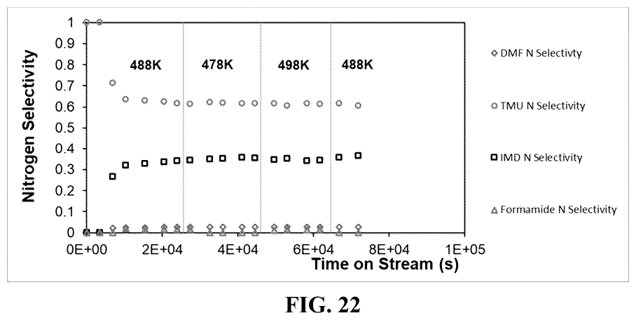

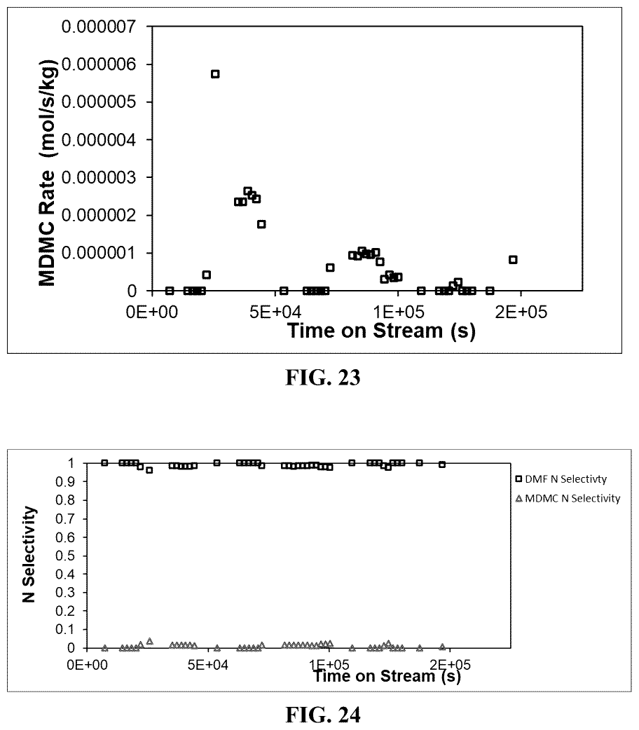

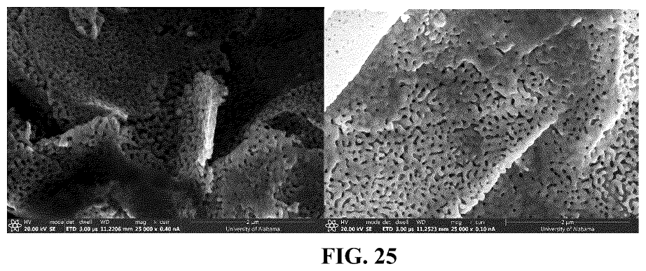

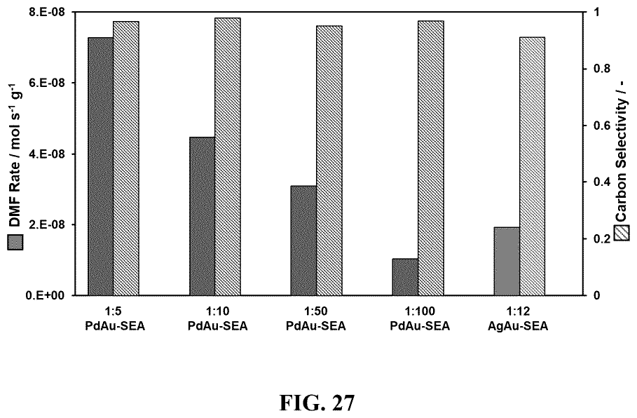

Results characterizing the formation of DMF on heterogeneous metallic catalysts are shown in FIGS. 5-11. FIGS. 12-15 and 25 include SEM/TEM images characterizing the nanostructure of catalysts supported on silica. FIGS. 16A-16B, 17A-17B, 18A-18B, 19A-19B, 20A-20B, 21A-21B, and 27 depict DMF production rates and selectivity for several types of heterogeneous metallic catalysts synthesized during this experiment. FIGS. 22 and 24 include plots showing N₂ selectivity of several products over PdAu/SiO₂ and 1:15 AgAu/SiO₂ catalysts, respectively. FIG. 23 includes an evolution rate of methyl dimethylcarbamate (MDMC) at elevated temperature (423K-473K) over a 1:15 AgAu/SiO₂ catalyst.

TABLE 2
Summary of catalytic data for each sample.

| | Total Metal | | | DMF Rate | |
|---|---|---|---|---|---|
| | Weight | Metal | DMF Rate | (mol (mol | DMF Carbon |
| Catalyst | Loading | Molar | (mol g_{catalyst}⁻¹ | dilute | Selectivity |
| Composition | (wt %) ^{a, b} | Ratio ^{a, b} | s⁻¹) | metal)⁻¹ s⁻¹) | (unitless)^{c} |
| Au/SiO₂ | 20 ^{b} | — | 0 | 0 | 0 |
| Pd/SiO₂ | 0.6 ^{b} | — | 1.7 × 10⁻⁹ | — | 0.45 |
| NiCu/SiO₂ | 8 ^{b} | 1:100 ^{b} | 1.1 × 10⁻⁹ | 8.4 × 10⁻⁵ | 0.39 |
| PdCu/SiO₂ | 8 ^{b} | 1:50 ^{b} | 1.1 × 10⁻⁹ | 4.4 × 10⁻⁵ | 0.30 |
| AgAu/SiO₂ | 3.5 ^{b} | 1:15 ^{b} | 2.3 × 10⁻⁹ | 7.1 × 10⁻⁵ | 0.70 |
| NiAu/SiO₂ | 5 | 1:10 | 2.4 × 10⁻⁸ | 8.9 × 10⁻⁴ | 0.77 |
| PtAu/SiO₂ | 4 | 1:50 | 1.4 × 10⁻⁸ | 3.4 × 10⁻³ | 0.83 |
| PdAu/SiO₂ | 2.5 ^{a} | 1:2 ^{a} | 3.5 × 10⁻⁸ | 1.7 × 10⁻⁴ | 0.75 |
| PdAu/SiO₂ | 3.5 ^{a} | 1:10 ^{a} | 3.7 × 10⁻⁸ | 1.1 × 10⁻³ | 0.79 |
| PdAu/SiO₂ | 2.3 ^{b} | 1:53 ^{b} | 4.3 × 10⁻⁸ | 1.3 × 10⁻² | 0.96 |
| PdAu/SiO₂ | 4 ^{b} | 1:83 ^{b} | 1.5 × 10⁻⁸ | 6.5 × 10⁻³ | 0.92 |

^{a} Weight loadings and molar ratios measured by ICP-OES.
^{b} Weight loadings and molar ratios based on synthesis protocol.
^{c}Conservative estimate for DMF selectivity based on detection of CO₂ by GC-MS. No CO₂ was detected by GC-TCD, thus the DMF selectivity may be higher in all cases.

### REFERENCES

Roughley, S. D., Jordan, Allan M., The Medicinal Chemist's Toolbox: An Analysis of Reactions Used in the Pursuit of Drug Candidates. Journal of Medicinal Chemistry, 2011. 54(10): p. 3451-3479.
Seavill, P. W., Wilden, Jonathan D., The Preparation and Applications of Amides Using Electrosynthesis. Green Chemistry, 2020. 22: p. 7737-59.
N,N-Dimethylformamide. 2021 May 3, 2021]; Available from: pubchem.ncbi.nlm.nih.gov/compound/N_N-Dimethylformamide
Bipp H, K. H., Formamides, in Ullmann's Encyclopedia of Industrial Chemistry. 2011, John Wiley & Sons.: New York, NY.
Haynes, P. K., Josef F.; Slaugh, Lynn H., Formamide production, U. States, Editor. 1970, Shell Oil Co: US.
Couteau, W. R., Jean, Process for the production of formamides, U. States, Editor. 1978, UCB SA: US.
Nozaki, K., Production of n,n-dimethylformamide, US, Editor. 1969, Shell Oil Co: United States.
Siefen, H. T. T., William R., Low pressure production of formamides, U. States, Editor. 1958, EI Du Pont de Nemours and Co: US.
Bellis, H. E., Method for producing monomethylformamide and dimethylformamide, U. States, Editor. 1989, EI Du Pont de Nemours and Co: US.
Maliszewskyj, R. J. T., Michael Gerard; Mitchell, John William, Dimethylformamide synthesis via reactive distillation of methyl formate and dimethylamine, U. States, Editor. 2004, Air Products and Chemicals Inc: US.
Clemens, S. R., Preparation of dimethylformamide, U. States, Editor. 1963, EI Du Pont de Nemours and Co: US.
Baker E. G., M. L. K., Brown M. D., Methanol and Ammonia From Biomass: Laboraory-scale gasifier tests show that catlytic gasification of biomass to produce methanol and ammonia synthesis gases is technically feasible. Chemical Engineering Progress, 1984. 80(12): p. 43-46.
Nakagawa H., H. T., Ichinose T., Takeno K., Matsumoto S., Kobayashi M., Sakai M., Biomethanol Production and CO₂ Emission Reduction from Forage Grasses, Trees, and Crop Residues. Japan Agricultural Research Quarterly: JARQ, 2007. 41(2): p. 173-180.
Williams R. H., L. E. D., Katofsky R. E., Chen J., Methanol and hydrogen from biomass for transportation. Energy for Sustainable Development, 1995. 1(5): p. 18-34.
Omura, K., Swern, Daniel, Oxidation of alcohols by “activated” dimethyl sulfoxide.
a preparative, steric and mechanistic study. Tetrahedron, 1978. 34(11): p. 1651-1660.
Chebolu, R., Bahuguna, Ashish, Sharma, Reena, Mishra, V. K., Ravikumar, P. C., An unusual chemoselective oxidation strategy by an unprecedented exploration of an electrophilic center of DMSO: a new facet to classical DMSO oxidation. Chemical Communications, 2015. 51(84): p. 15438-15441.
Zhao M., L. J., Song Z., Desmond R., Tschaen D. M., Grabowski E. J. J., Reider P. J., A novel chromium trioxide catalyzed oxidation of primary alcohols to the carboxylic acids. Tetrahedron Letters, 1998. 39(30): p. 5323-5326.
Xu B., M. R. J., Friend C. M., Predicting Gold-Mediated Cataytic Oxidative-Coupling Reaction from Single Crystal Studies. Accounts of Chemical Research, 2013. 47(3): p. 731-772.
Arne Wittstock, B. N., Andreas Schaefer, Karifala Dumbuya, Christian Kilbel, Monika Biener, Volkmar Zielasek, Hans-Peter Steinrück, J. Mickael Gottfried, Jürgen Beiner, Alex Hamza, Marcus Bäumer, Nanoporous Au: An Unsupported Pure Gold Catalyst? The Journal of Physical Chemistry C, 2009. 113(14): p. 5593-5600.
A. Wittstock, V. Z., J. Biener, C. M. Friend, M. Bäumer, Nanoporous Gold Catalysts for Selective Gas-Phase Oxidative Coupling of Methanol at Low Temperature. Science, 2010. 327(5963): p. 319-322.
Personick M., Z. B., Monika B., Juergen B., Madix R. J., Friend C. M., Ozone-Activated Nanoporous Gold: A Stable and Storable Material for Catalytic Oxidation. ACS Catalysis, 2015. 5(7): p. 4237-4241.
Lackmann A, M. C., Schowalter M., Fitzek L., Weissmüller J., Rosenauer A.,
Wittstock A., A comparative study of alcohol oxidation over nanoporous gold in gas and liquid phase. Journal of Catalysis, 2017(353): p. 99-106.
Reece C., L. M., Madix R. J., Dissecting the Performance of Nanoporous Gold Catalysts for Oxygen-Assisted Coupling of Methanol with Fundamental Mechanistic and Kinetic Information. ACS Catalysis, 2019. 9(5): p. 4477-4487.
Personick M., M. R. J., Friend C. M., Selective Oxygen-Assisted Reactions of Alcohols and Amines Catalyzed by Metallic Gold: Paradigms for the Design of Catalytic Processes. ACS Catalysis, 2017. 7(2): p. 965-985.
Wichmann, A., Marcus Baumer, and Arne Wittstock, Oxidative Coupling of Alcohols and Amines over Bimetallic Unsupported Nanoporous Gold: Tailored Activity through Mechanistic Predictability. ChemCatChem, 2014. 7: p. 70-74.
Xu B., H. J., Freyschlag C. G., Madix, R. J., Friend C. M., Oxygen-assisted cross-coupling of methanol with alkyl alcohols on metallic gold. Chemical Science, 2010. 1(3): p. 310-314.
Xu B., X. L., Haubrich J., Madix R. J., Friend C. M., Selectivity Control in Gold-Mediated Esterification of Methanol. Angewandte Chemie, 2009. 121(23): p. 4270-4273.
Bingjun Xu, L. Z., Robert J. Madix, Cynthia M. Friend, Highly Selective Acylation of Dimethylamine Mediated by Oxygen Atoms on Metallic Gold Surfaces. A Journal of the German Chemical Society 2012. 49(2): p. 394-398.
Zhou, L., Freyschlag, Cassandra G., Xu, Bingjun, Friend, Cynthia M., Madix, Robert J., Direct selective oxygen-assisted acylation of amines driven by metallic silver surfaces: dimethylamine with formaldehyde. Chemical Communications, 2010. 46(5): p. 704-706.
Xu B., H. J., Baker T. A., Kaxiras E., Friend C. M., Theoretical Study of 0-Assisted Selective Coupling of Methanol on Au(111). The Journal of Physical Chemistry C, 2011. 115(9): p. 3703-3708.
Xu, B. M. R. J. F. C. M., Achieving Optimum Selectivity in Oxygen Assisted Alcohol Cross-Coupling on Gold. Journal of the American Chemical Society, 2010. 132(46): p. 16571-16580.
Montemore, M. A. v. S., M. A.; Madix, R. J.; Friend, C. M., O₂ Activation by Metal Surfaces: Implications for Bonding and Reactivity on Heterogeneous Catalysts. Chemical Reviews, 2018. 118(5): p. 2816-2862.
Yu, W.-Y., Liang Zhang, Gregory M. Mullen, Graeme Henkelman, and C. Buddie Mullins, Oxygen Activation and Reaction on Pd-Au Bimetallic Surfaces. The Journal of Physical Chemistry, 2015. 119(21): p. 11754-62.
Klobas, E., J; Schmid, M.; Friend, C. M.; Madix, R. J., The dissociation-induced displacement of chemisorbed O₂ by mobile O atoms and the autocatalytic recombination of O due to chain fragmentation on Ag(110). Surface Science, 2014. 630: p. 187-194.
Chang, C.-R. L., Bo; Yang, Xiao-Feng; Li Jun, Theoretical Studies on the Synergetic Effects of Au-Pd Bimetallic Catalysts in the Selective Oxidation of Methanol. The Journal of Physical Chemistry C. 119(28): p. 16072-16081.
Filie, A. S., T.; Foucher, A. C.; Stach, E. A.; Aizenburg, M.; Aizenburg J.; Friend, C. M.; Madix, R. J., Dilute Pd-in-Au alloy RCT-SiO₂ catalysts for enhanced oxidative methanol coupling. Journal of Catalysis, 2021. 404: p. 943-953.
Junjun Shan, G. G., Jilei Liu, Sufeng Cao, Mengyao Ouyang, Mengwei Li, Sungsik Lee, Maria Flytzani-Stephanopoulos, PdCu Single Atom Alloys for the Selective Oxidation of Methanol to Methyl Formate at Low Temperatures. Topics in Catalysis, 2020. 63(7): p. 618-627.
Zhang, Q. Z., C.; Yang, G.; Sun, Y.; Wang, D.; Liu, J., High-performance micro structured Au—Ag bimetallic catalyst for oxidative coupling of methanol to methyl formate. Catalysis Communications, 2019. 129: p. 105741.
Liu, X., Aiqin Wang, Xiaofeng Yang, Tao Zhang, Chung-Yuan Mou, Dang-Sheng Su, and Jun Li, Synthesis of Thermally Stable and Highly Active Bimetallic Au-Ag Nanoparticles on Inert Supports. Chemistry of Materials, 2009. 21(2): p. 410-18.
Liu, X., Aiqin Wang, Lin Li, Tao Zhang, Chung-Yuan Mou, and Jyh-Fu Lee, Synthesis of Au—Ag Alloy Nanoparticles Supported on Silica Gel via Galvanic Replacement Reaction. Progress in Natural Science: Materials International, 2013. 23(3): p. 317-25.
Jilei Liu, J. S., Felicia R. Lucci, Sufeng Cao, Charles Sykes, Maria Flytzani-Stephanopoulo, Palladium-gold single atom alloy catalysts for liquid phase selective hydrogenation of 1-hexyne. Catalysis Science & Technology, 2017. 7(19): p. 4276-4284.
Dong, A. S., A.; Karakalos, S.; Blom, D.; Williams, C. T.; Regalbuto. J. R., The preparation of silica supported, dilute limit PdAu alloys via simultaneous strong electrostatic adsorption. Catalysis Science & Technology, 2023. 13(10): p. 3020-3034.
Mathilde Luneau, E. G., Wei Chen, Alexandre C. Foucher, Nicholas Marcella, Tanya Shirman, David M. A. Berbart, Joanna Aizenburg, Michael Aizenburg, Eric A. Stach, Robert J. Madix, Anatoly I. Frenkel, Cynthia M. Friend., Enhancing catalytic performance of dilute metal alloy nanomaterials. Communications Chemistry, 2020. 3(1): p. 1-9.

The methods and compositions of the appended claims are not limited in scope by the specific methods and compositions described herein, which are intended as illustrations of a few aspects of the claims and any methods and compositions that are functionally equivalent are within the scope of this disclosure. Various modifications of the methods and compositions in addition to those shown and described herein are intended to fall within the scope of the appended claims. Further, while only certain representative methods, compositions, and aspects of these methods and compositions are specifically described, other methods and compositions and combinations of various features of the methods and compositions are intended to fall within the scope of the appended claims, even if not specifically recited. Thus, a combination of steps, elements, components, or constituents can be explicitly mentioned herein; however, all other combinations of steps, elements, components, and constituents are included, even though not explicitly stated.

What is claimed is:

1. A method of forming an N,N-substituted-formamide comprising:
contacting a feed gas comprising an alcohol and an N,N-substituted-amine with a solid heterogeneous catalyst in the presence of an oxidizing agent at a reaction temperature, wherein the solid heterogeneous catalyst comprises one or more noble metals; and
oxidatively coupling the feed gas under conditions effective to form an effluent gas comprising the N,N-substituted-formamide.

2. The method of claim 1, wherein the solid heterogeneous catalyst is disposed on a support.

3. The method of claim 1, wherein the solid heterogenous catalyst comprises nanoparticles, and/or a nanoporous material.

4. The method of claim 1, wherein the solid heterogeneous catalyst comprises palladium.

5. The method of claim 1, wherein the solid heterogeneous catalyst comprises gold.

6. The method of claim 1, wherein the solid heterogeneous catalyst comprises a bimetallic catalyst.

7. The method of claim 6, wherein the solid bimetallic catalyst comprises a Pd/Au alloy.

8. The method of claim 6, wherein the solid bimetallic catalyst has an atomic ratio of Pd to a second metal of from 100:1 to 1:1000.

9. The method of claim 1, wherein the N,N-substituted-amine comprises dimethylamine (DMA), the alcohol comprises methanol, and the N,N-substituted-formamide comprises dimethylformamide (DMF).

10. The method of claim 1, wherein the reaction temperature is from 75° C. to 250° C.

11. A method of forming a carbamide and/or a carbamate comprising:
contacting a feed gas comprising an alcohol and an N,N-substituted-amine with a solid heterogeneous catalyst in the presence of an oxidizing agent at a temperature greater than or equal to 155° C., wherein the solid heterogeneous catalyst comprises one or more noble metals; and
oxidatively coupling the feed gas under conditions effective to form a product gas comprising the carbamide and/or carbamate.

12. The method of claim 11, wherein the solid heterogeneous catalyst is disposed on a support.

13. The method of claim 11, wherein the solid heterogeneous catalyst comprises nanoparticles, and/or a nanoporous material.

14. The method of claim 11, wherein one or more noble metals are chosen from gold (Au), palladium (Pd), silver (Ag), platinum (Pt), copper (Cu), and combinations thereof.

15. The method of claim 11, wherein the solid heterogeneous catalyst comprises palladium.

16. The method of claim 11, wherein the solid heterogeneous catalyst comprises gold.

17. The method of claim 11, wherein the solid heterogeneous catalyst comprises a solid bimetallic catalyst.

18. The method of claim 17, wherein the solid bimetallic catalyst comprises a Pd/Au alloy and/or an Ag/Au alloy.

19. The method of claim 11, wherein the N,N-substituted-amine comprises dimethylamine (DMA).

20. The method of claim 11, wherein the carbamide and/or a carbamate comprises tetramethyl urea (TMU), imidazolida-2-one, methyldimethylcarbamate (MDMC), or combinations thereof.
